(19) United States
(12) Patent Application Publication
(10) Pub. No.: US 2024/0198115 A1
(43) Pub. Date: Jun. 20, 2024

(54) CONNECTOR CONTACT FOR ELECTRICAL STIMULATION SYSTEM

(71) Applicant: Boston Scientific Neuromodulation Corporation, Valencia, CA (US)

(72) Inventor: Bernard Zdzislaw Malinowski, Castaic, CA (US)

(21) Appl. No.: 18/540,469

(22) Filed: Dec. 14, 2023

Related U.S. Application Data
(60) Provisional application No. 63/432,630, filed on Dec. 14, 2022.

(51) Int. Cl. A61N 1/375; H01R 13/631; H01R 24/58

(52) U.S. Cl. CPC A61N 1/3752; H01R 13/6315; H01R 24/58

(57) ABSTRACT

A system for making an electrical connection to a lead contact may include a connector contact assembly. The assembly may include a connector block configured to receive and retain a conductive spring within an interior of the block. The block may have an open center portion and first and second ends with first and second flanges. The spring may be formed from a material with shape-memory, and may be configured to be compressed for insertion through one of the flanges, and to at least partially expand into an expanded state such that the spring is retained between the flanges. The spring may have a biasing structure to physically contact the lead contact. The system is capable of accommodating misaligned connectors, as each conductive spring is able to position itself concentrically with a lead body without stretching or deforming.

### CLAIM OF PRIORITY

This application claims the benefit of U.S. Provisional Application No. 63/432,630, filed on Dec. 14, 2022, which is hereby incorporated by reference in its entirety.

### TECHNICAL FIELD

This document relates generally to medical systems, and more particularly, but not by way of limitation, to systems, devices, and methods for using electrical stimulation systems having improved connector contacts.

### BACKGROUND

Medical devices may include therapy-delivery devices configured to deliver a therapy to a patient and/or or monitors configured to monitor a patient condition via user input and/or sensor(s). For example, therapy-delivery devices for ambulatory patients may include wearable devices and implantable devices, and further may include, but are not limited to, stimulators. An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc.

A stimulator can include a control module (with a waveform generator such as a pulse generator), one or more leads, and an array of stimulator electrodes on each lead. The stimulator electrodes are in contact with or near the nerves, muscles, or other tissue to be stimulated. The waveform generator generates an electrical waveform that is delivered by the electrodes to body tissue. A pulse generator generates electrical pulses that are delivered by the electrodes to body tissue.

A medical device may use a plurality of connectors to make an electrical connection to a plurality of lead contacts on a proximal end of a lead. For example, the proximal end may be inserted into a connector stock that contains the connectors. However, the connector stock may include misaligned connectors, which may cause the contacts within the misaligned connectors to be stretched, deformed or otherwise be damaged when the proximal end of the lead is inserted.

### SUMMARY

Various embodiments provide a connector design capable of being used with misaligned connectors, as the connectors have some allowable movement enabling each connector to position itself concentrically with the lead body when the proximal end of the lead is inserted. The design avoids stretching, deforming or otherwise damaging the contacts within the misaligned connectors when the lead is inserted.

An example (e.g., Example 1) of a system for making an electrical connection to a lead contact of a medical device lead or lead extension may include a connector contact assembly. The connector contact assembly may include a connector block configured to receive and retain a conductive spring within an interior of the connector block to form the contact connector. A combination of the connector block and the spring is configured to receive a proximal end of the medical lead or a proximal end of the lead extension when the proximal end is inserted through the combination. The connector block may have an open center portion, and further may have a first end, an opposing second end, a longitudinal length extending between the first end and the second end, an outer surface, and an inner surface forming a perimeter of the open center portion of the connector block. The connector block may further include a first flange at the first end and a second flange at the second end. The first and second flanges may extend inward from the inner surface of the connector block. The spring may be formed from a material with shape-memory, and may be configured to be compressed for insertion through one of the first flange or the second flange, and to at least partially expand into an expanded state such that the spring is retained between the first flange and the second flange within the open center portion of the connector block. The spring may have a biasing structure configured to physically contact the lead contact when the proximal end is inserted into a final position within the connector contact assembly.

In Example 2, the subject matter of Example 1 may optionally be configured such that both the connector block and the spring are conductive and in contact with each other, and a conductive member extends from an exterior of the connector block to provide an electrical connection to the spring retained within the open center portion of the connector block.

In Example 3, the subject matter of any one or more of Examples 1-2 may optionally be configured such that the spring is not affixed to the connector block, and the combination of the connector block and the spring is configured to allow movement of the spring, when the spring is in the expanded state between the first flange and the second flange, within the open center portion of the connector block.

In Example 4, the subject matter of any one or more of Examples 1-3 may optionally be configured such that when the spring is retained within the open center portion of the connector block, the connector block has a connector block center axis and the spring has a spring center axis generally parallel to the connector block, and the movement moves an offset of the spring center axis with respect to the connector block axis to accommodate the lead.

In Example 5, the subject matter of Example 4 may optionally be configured such that the spring includes a first end and an opposing second end, and a first rim at the first end and a second rim at the second end. Each of the first and second rims have rim ends and a gap separating the rim ends. The biasing structure includes a plurality of biasing members configured to physically contact that lead contact. Each of the plurality of biasing members extend between the first rim and the second rim and include a bend to provide a biasing force against the lead contact. The biasing forces corresponding to the plurality of biasing members align the spring center axis with a center axis of the proximal portion of the lead or the lead extension, such that the spring positions itself concentrically with the proximal end of the lead or proximal end of the lead extension.

In Example 6, the subject matter of Example 5 may optionally be configured such that each of the plurality of biasing members are perpendicularly attached to the first rim and to the second rim.

In Example 7, the subject matter of any one or more of Examples 5-6 may optionally be configured such that the first and second rims are arc-shaped.

In Example 8, the subject matter of any one or more of Examples 5-7 may optionally be configured such that each of the first and second rims has an angle of rotation greater than 240 degrees and the gap has an angle of rotation less than 120 degrees. Each of the first and second rims may have an angle of rotation which may be greater than 270 degrees and the gap may have an angle of rotation less than 90 degrees. Each of the first and second rims may have an angle of rotation which may be greater than 315 degrees and the gap may have an angle of rotation less than 45 degrees. Each of the first and second rims may have an angle of rotation between 320 to 340 degrees and the gap may have an angle of rotation between 20 to 40 degrees.

In Example 9, the subject matter of any one or more of Examples 1-8 may optionally be configured such that both the connector block and the spring are made from a same conductive material. Benefits for using the same conductive material includes assuring conductivity and avoiding ionic corrosion.

In Example 10, the subject matter of any one or more of Examples 1-9 may optionally be configured such that the biasing structure comprises at least one bend that extends into the open center portion of the contact housing and narrows an inner diameter of the open center portion.

In Example 11, the subject matter of any one or more of Examples 1-10 may optionally be configured such that the connector block has a cylindrical shape, where both the outer surface and the inner surfaces are curved surfaces.

In Example 12, the subject matter of any one or more of Examples 1-11 may optionally be configured such that the connector-contact assembly is one of a plurality of similar connector-contact assemblies, and the system further includes an elongated connector housing having a first end and an opposing second end, a connector lumen defined in the connector housing, the connector lumen configured and arranged to receive the proximal end of the lead or the lead extension, and the plurality of similar connector-contact assemblies.

In Example 13, the subject matter of Example 12 may optionally be configured such that when the spring is retained within the interior of the connector block, the connector block has a connector block center axis and the spring has a spring center axis generally parallel to the connector block, and the movement moves an offset of the spring center axis with respect to the connector block axis to accommodate the lead. At least two connector blocks within the plurality of connector-contact assemblies have connector block axes misaligned with respect to each other. The combination of the connector block and the spring for each of the plurality of connector-contact assemblies are configured to allow movement of the spring in the expanded state within the open center portion of the connector block when the spring is retained between the first flange and the second flange such that a spring axis aligns with a center axis of the proximal end of the lead or the lead extension.

In Example 14, the subject matter of any one or more of Examples 12-13 may optionally be configured to further include a medical device having a control module configured to be electrically connected to the lead.

An example (e.g., Example 15) of a medical device may include a control module configured to be electrically connected to a lead. The lead may include a lead body with a proximal portion, a distal portion, a longitudinal length, a plurality of electrodes disposed along the distal portion of the lead body, a plurality of terminals disposed along the proximal portion of the lead body, and a plurality of lead conductors electrically coupling the plurality of electrodes to the plurality of terminals. The control module may include a housing, an electronic subassembly disposed in the housing, an elongated connector housing having a first end and an opposing second end, a connector lumen defined in the connector housing where the connector lumen is configured and arranged to receive the proximal end of the lead or the lead extension, and a plurality of similar connector-contact assemblies. Each of the plurality of connector-contact assemblies may include any one or more of the connector-contact assemblies provided in the systems of Examples 1-14.

An example (e.g., Example 16) of a method may include assembling a connector contact assembly for use to make an electrical connection to a lead contact of a medical device lead or lead extension. The connector contact assembly may include a connector block configured to receive and retain a conductive spring having shape memory within an interior of the connector block. The connector block may have a first open end, an opposing second open end, a longitudinal length extending between the first open end and the second open end, an inner surface, an outer surface, a first flange at the first open end and a second flange at the second open end where the first and second flanges extend past the inner surface toward the interior of the connector block. The assembling the connector contact assembly may include applying a compressive force to the spring to reduce a spring size to a compressed size, inserting the spring having the compressed size through an opening in a first flange or opening in a second flange so that the spring is with the interior of the connector block, and removing the compressive force, allowing the spring to expand from the compressed shape to at least a partially expanded shape larger than the opening to the first flange or the second flange such that the spring is retained within the interior of the connector block. The connector-contact assembly may include any one or more of the connector-contact assemblies provided in the systems of Examples 1-14.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIGS. 1A-1B illustrate, by way of example and not limitation, various embodiments of an electrical stimulation system.

FIGS. 2A, 2B and 2C illustrates, by way of example and not limitation, various applications for connectors.

FIGS. 3A-3B illustrate views of an assembled connector contact assembly, by way of example and not limitation.

FIG. 4A illustrates a connector block, FIG. 4B illustrates a spring, and FIG. 4C illustrates a gap within a circular rim of the spring, by way of examples and not limitation.

FIG. 5 illustrates, by way of example and not limitation, an assembly of a spring into a connector block to form a connector contact assembly.

FIG. 6 illustrates, by way of example and not limitation, a cutaway view of the assembled contact connector.

FIG. 7 illustrates, by way of example and not limitation, a connector with eight connector contact assemblies.

FIG. 8 illustrates, by way of example and not limitation, a conductive member attached to the exterior of the conductor block for use to provide an electrical connection to the spring.

FIG. 9 illustrates a proximal end of a lead and a plurality of connector contact assemblies through which the lead is inserted.

FIG. 10 illustrates, by way of example and not limitation, a view from an end of the header having four lumens for receiving four leads into four connectors, and showing center offsets for the center axis (illustrated using “+”) of at least two of the connector blocks used in the connector contact assemblies for each connector.

FIG. 11 illustrates, by way of example and not limitation, a lead with a center axis and a circular lead profile.

FIG. 12 illustrates, by way of example and not limitation, an offset between the center axes of a connector block and lead, and the accommodation of the offset by movement of the spring into alignment with the lead.

FIGS. 13A-13C illustrate, by way of example and not limitation, potential spring motion within the connector block to accommodate the offset.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

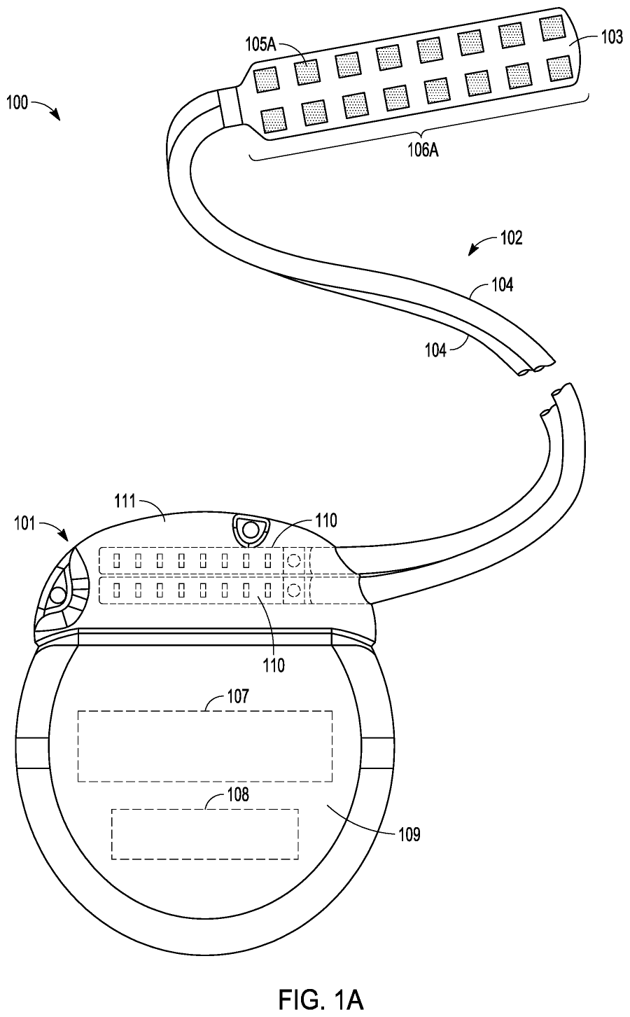

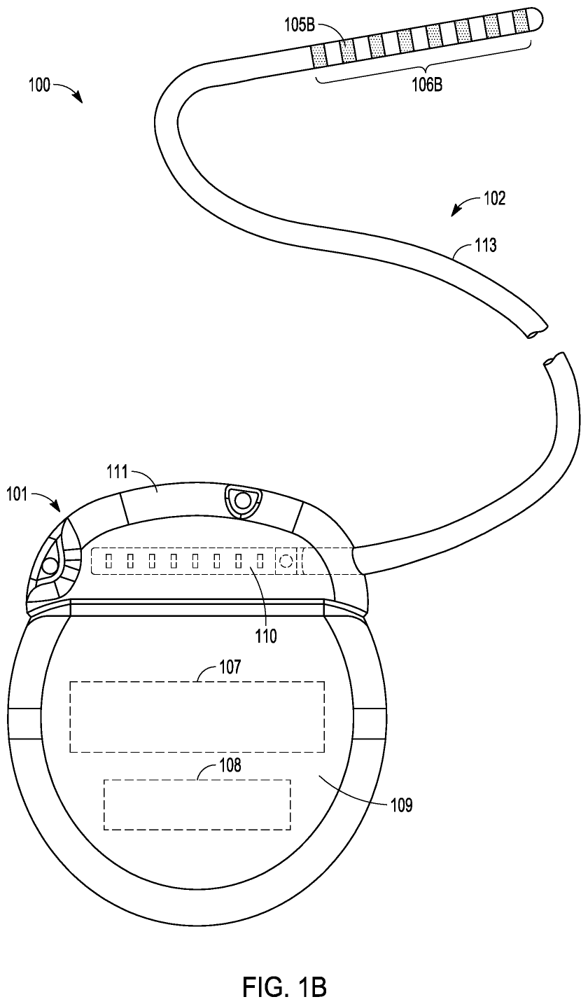

FIGS. 1A-1B illustrate, by way of example and not limitation, various embodiments of an electrical stimulation system 100. The electrical stimulation system 100 may include a control module (e.g., a stimulator or pulse generator) 101 and a lead 102. As illustrated in FIG. 1A, the lead 102 may include a paddle body 103 and one or more lead bodies 104 coupling the control module 101 to the paddle body 103. The paddle body 103 and the one or more lead bodies 104 form the lead 102. The paddle body 103 may include a plurality of electrodes 105B that form an array of electrodes 106B. The control module 101 may include an electronic subassembly 107 and a power source 108 disposed in a sealed housing 109. In FIG. 1A, two lead bodies 104 are shown coupled to the control module 101.

The control module 101 may include one or more connectors 110 into which the proximal end of the one or more lead bodies 104 can be plugged to make an electrical connection via connector contacts disposed in the connector 110 and terminals on each of the one or more lead bodies 104. The connector contacts are coupled to the electronic subassembly 107 and the terminals are coupled to the electrodes 105. In FIG. 1A, two connectors 110 are shown.

The one or more connectors 110 may be disposed in a header 111 that provides a protective covering over the one or more connectors 110. The header 111 may be formed using any suitable process including, for example, casting, molding (including injection molding), and the like. In addition, one or more lead extensions can be disposed between the one or more lead bodies 104 and the control module 101 to extend the distance between the one or more lead bodies 104 and the control module 101.

Those of ordinary skill in the art, who have read and understood this document, will understand that the electrical stimulation system can include more, fewer, or different components and can have a variety of different configurations including those configurations disclosed in the electrical stimulation system references cited herein. For example, instead of a paddle body 103, the electrodes 105B may be disposed in an array 106B at or near the distal end of a lead body 112′ forming a percutaneous lead 113, as illustrated in FIG. 1B. The percutaneous 113 lead may be isodiametric along the length of the lead body 113. The lead body 112 may be coupled with a control module 101 with a single connector 110.

The electrical stimulation system or components of the electrical stimulation system, including one or more of the lead bodies, the control module, and, in the case of a paddle lead, the paddle body, may be implanted into the body of a patient. The electrical stimulation system may be configured to be used for a variety of applications including, but not limited to, spinal cord stimulation, brain stimulation, neural stimulation, muscle activation via stimulation of nerves innervating muscle, and the like.

The electrodes 105A and 105B may be formed using any conductive, biocompatible material. Examples of suitable materials include metals, alloys, conductive polymers, conductive carbon, and the like, as well as combinations thereof. In at least some embodiments, one or more of the electrodes 105A and 105B are formed from one or more of: platinum, platinum iridium, palladium, titanium, or rhenium.

The number of electrodes 105A and 105B in the array of electrodes 106A and 106B may vary. For example, there can be two, three, four, five, six, seven, eight, nine, ten, eleven, twelve, thirteen, fourteen, fifteen, sixteen, or more electrodes. As will be recognized, other numbers of electrodes may also be used. The electrodes 105A and 105B may be formed in any suitable shape including, for example, round, oval, triangular, rectangular, pentagonal, hexagonal, heptagonal, octagonal, or the like.

The electrodes of the paddle body or one or more lead bodies are typically disposed in, or separated by, a non-conductive, biocompatible material including, for example, silicone, polyurethane, and the like or combinations thereof. The paddle body and one or more lead bodies may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead to the proximal end of each of the one or more lead bodies. The non-conductive, biocompatible material of the paddle body and the one or more lead bodies may be the same or different. The paddle body 104 and the one or more lead bodies may be a unitary structure or can be formed as two separate structures that are permanently or detachably coupled together.

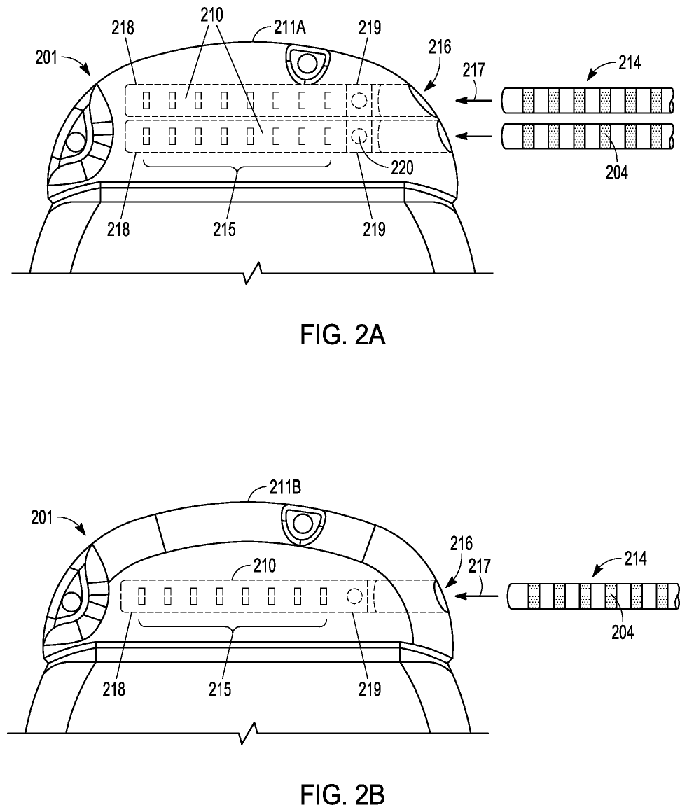

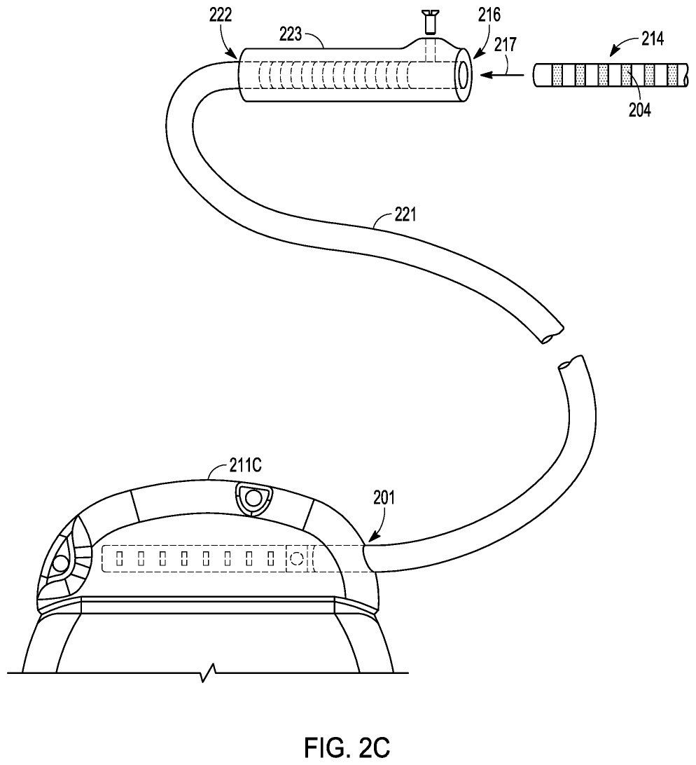

FIGS. 2A, 2B and 2C illustrates, by way of example and not limitation, various applications for connectors. FIG. 2A illustrates a header 211A that includes at least two connector lumens with a plurality of connector-contact assemblies disposed therein to receive at least two proximal lead ends, FIG. 2B illustrates a header 211B that includes a connector lumen with a plurality of connector-contact assemblies disposed therein to receive a proximal lead end, and FIG. 2C illustrates a header 211C that includes a connector lumen with a plurality of connector-contact assemblies disposed therein to receive a proximal end of a lead extension.

Terminals 214 are typically disposed at the proximal end of the one or more lead bodies 204 for connection to corresponding connector contact assemblies 215 in connectors 210 disposed on, for example, the control module 201 (or to other devices, such as conductive contacts on a lead extension, an operating room cable, a splitter, an adaptor, or the like). Conductive wires (not shown) extend from the terminals 214 to the electrodes 105A, 105B. Typically, one or more electrodes 105A, 105B are electrically coupled to a terminal 214. In some embodiments, each terminal 214 is only coupled to one electrode 105A, 105B.The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the paddle body. The one or more lumens may, optionally, be flushed continually, or on a regular basis, with saline, epidural fluid, or the like. The one or more lumens can be permanently or removably sealable at the distal end.

The control module 201 can include any suitable number of connectors 210 including, by way of example and not limitation, two three, four, five, six, seven, eight, or more connectors 210. It will be understood that other numbers of connectors 210 may be used instead.

FIG. 2A is a schematic side view of one embodiment of a plurality of connectors 210 disposed on the control module 201. In at least some embodiments, the control module 201 includes two connectors 210. In at least some embodiments, the control module 201 includes four connectors. The proximal ends of the plurality of lead bodies 204 are shown configured and arranged for insertion to the control module 201. FIG. 2B is a schematic side view of an embodiment of a single connector 210 disposed on the control module 201. In FIG. 2B, the proximal end of the single lead body 204 is shown configured and arranged for insertion to the control module 201.

The connectors 210 may be disposed in the header 211A, 211B. In at least some embodiments, the header 211A, 211B defines one or more lumens 216 into which the proximal end(s) of the one or more lead bodies with terminals can be inserted, as shown by directional arrows 217, in order to gain access to the connector contacts disposed in the one or more connectors 210.

Each of the one or more connectors 210 may include a connector housing 218 and a plurality of connector contact assemblies 215 disposed therein. The connector housing 218 may provide access to the plurality of connector contact assemblies 215 via the lumen 216. In at least some embodiments, one or more of the connectors 210 further includes a retaining element 219 configured and arranged to fasten the corresponding lead body to the connector when the lead body is inserted into the connector to prevent undesired detachment of the lead body from the connector. For example, the retaining element 219 may include an aperture 220 through which a fastener (e.g., a set screw, pin, or the like) may be inserted and secured against an inserted lead body.

When the one or more lead bodies are inserted into the one or more lumens, the connector contact assemblies are aligned with the terminals 214 disposed on the one or more lead bodies to electrically couple the control module 201 to the electrodes disposed at a distal end of the one or more lead bodies.

FIG. 2C illustrates an embodiment in which the electrical stimulation system includes one or more lead extensions for corresponding to one or more lead bodies. The one or more lead bodies 204 can be coupled to one or more lead extensions 221 which, in turn, are coupled to the control module 201. A lead extension connector is disposed at a distal end 222 of the lead extension 221. The lead extension connector includes a connector housing 223. The connector housing 223 defines at least one lumen 216 into which a proximal end of the lead body with terminals can be inserted, as shown by directional arrow 217. The lead extension connector also includes a plurality of connector contact assemblies. When the lead body 106′ is inserted into the lumen 330, the connector contacts 340 disposed in the connector contact assemblies are aligned with the terminals on the lead body to electrically couple the lead extension to the electrodes disposed at a distal end (not shown) of the lead body. The proximal end of a lead extension may be similarly configured and arranged as a proximal end of a lead body. The lead extension may include a plurality of conductive wires (not shown) that electrically couple the connector contacts to a terminal on a proximal end of the lead extension. The conductive wires disposed in the lead extension may be electrically coupled to a plurality of terminals (not shown) disposed on the proximal end of the lead extension. In at least some embodiments, the proximal end of the lead extension is configured and arranged for insertion into a lead extension connector disposed in another lead extension. In other embodiments, the proximal end of the lead extension is configured and arranged for insertion into the connector disposed on the control module. The control modules 201 can receive either lead bodies or lead extensions. The electrical stimulation system may include a plurality of lead extensions.

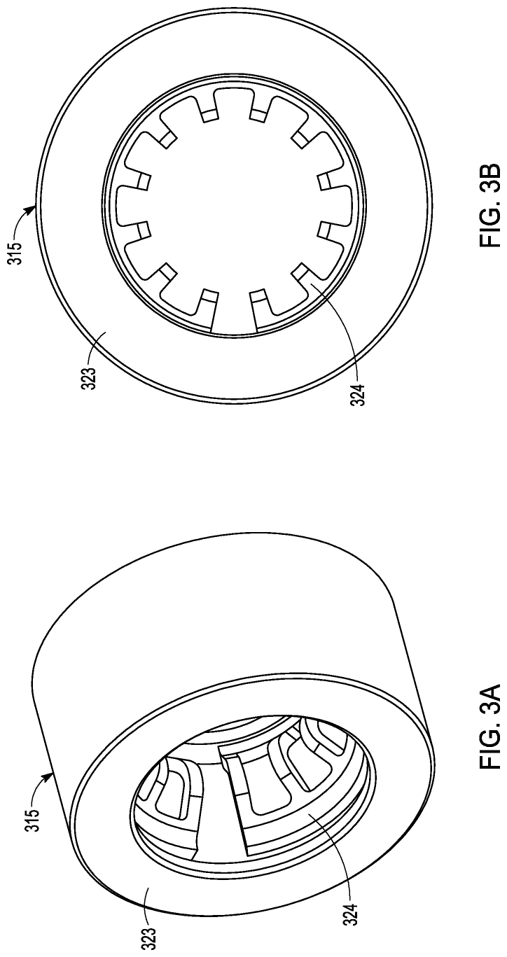

FIGS. 3A-3B illustrate views of an assembled connector contact assembly 315, by way of example and not limitation. The connector contact assembly 315 includes a connector block 323 configured to receive and retain a conductive spring 324 within an interior of the connector block 323 to form the connector contact assembly 315. A combination of the connector block 323 and the spring 324 is configured to receive a proximal end of the medical lead or a proximal end of the lead extension when the proximal end is inserted through the combination. The spring 324 is used to contact the terminals, such as terminals 214 illustrated in FIG. 2A-2C. As will be provided in more detail below, this design is an operational and structural improvement for contact connectors.

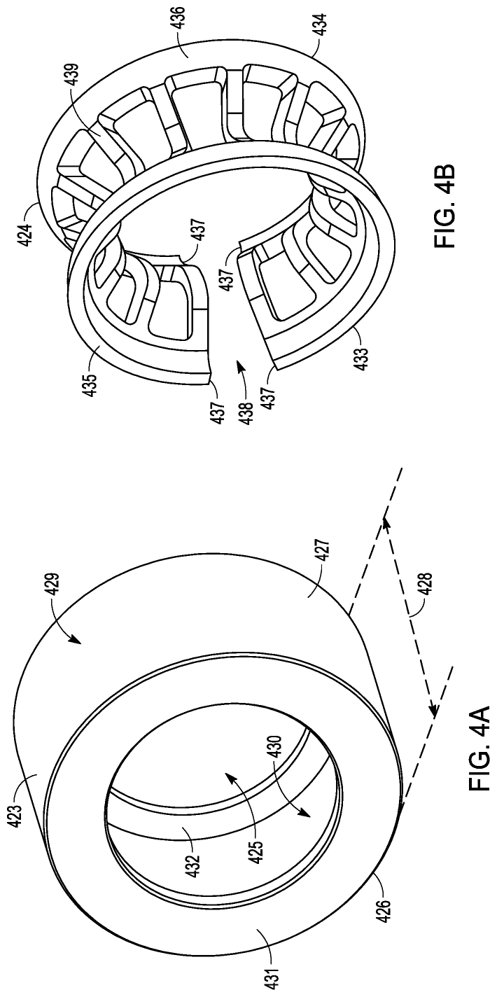

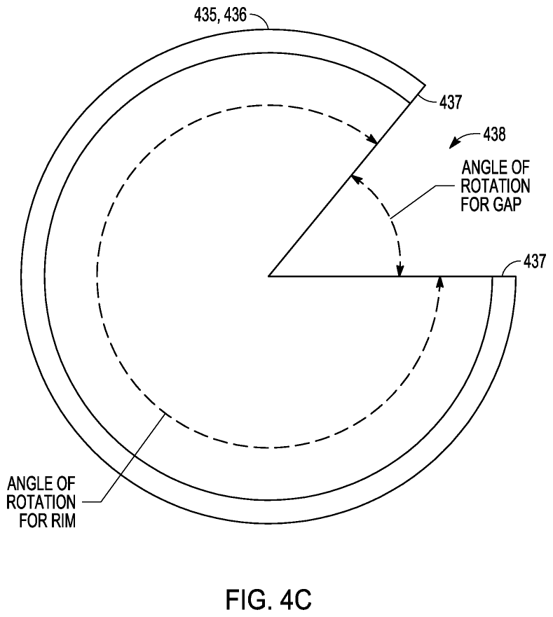

FIG. 4A illustrates a connector block, and FIG. 4B illustrates a spring, by way of examples and not limitation. The connector block 423 may have an open center portion 425, and further may have a first end 426, an opposing second end 427, a longitudinal length 428 extending between the first and second ends 426, 427, an outer surface 429, and an inner surface 430 forming a perimeter of the open center portion 425 of the connector block 423. The connector block 423 may have a cylindrical shape, where both the outer surface and the inner surfaces are curved surfaces. The connector block 423 may further include a first flange 431 at the first end 426 and a second flange 432 at the second end 427. The first and second flanges 431, 432 may extend inward from the inner surface of the connector block.

The spring 424 may be formed from a material with shape-memory, and may be configured to be compressed for insertion through one of the first flange 431 or the second flange 432, and to at least partially expand into an expanded state such that the spring 424 is retained between the first flange 431 and the second flange 432 within the open center portion 425 of the connector block 423. The spring 424 may have a biasing structure configured to physically contact the lead contact when the proximal end of the lead (or lead extension) is inserted into a final position within the connector contact assembly. The spring 424 is not welded or otherwise affixed to the connector block 423.

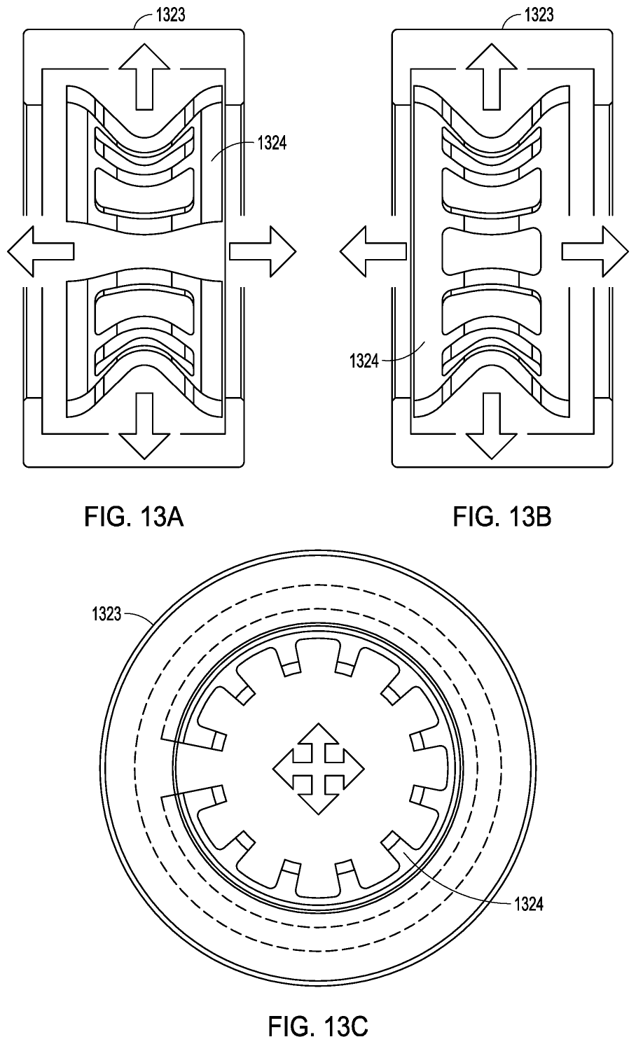

The combination of the connector block 423 and the spring 424 is configured to allow movement of the spring 424, when the spring is in the expanded state between the first flange 431 and the second flange 432, within the open center portion 425 of the connector block 423. When the spring is retained within the open center portion 425 of the connector block 423, the connector block has a connector block center axis and the spring has a spring center axis generally parallel to the connector block. The movement of the spring 424 moves an offset of the spring center axis with respect to the connector block axis to accommodate the lead, as generally illustrated in FIGS. 12, 13A, 13B and 13C. Thus, the spring positions itself concentrically with the proximal end of the lead or proximal end of the lead extension.

The spring 424 includes a first end 433 and an opposing second end 434, and a first rim 435 at the first end 433 and a second rim 436 at the second end 434. Each of the first and second rims 435, 436 have rim ends 437 and a gap 438 separating the rim ends 437. The biasing structure includes a plurality of biasing members 439 configured to physically contact that lead contact. Each of the plurality of biasing members 439 extend between the first rim 435 and the second rim 436 and include a bend to provide a biasing force against the lead contact. The bend in each of the biasing members extending to the open center portion of the contact housing and narrows an inner diameter of the open center portion. The biasing forces corresponding to the plurality of biasing members align the spring center axis with a center axis of the proximal portion of the lead or the lead extension. Each of the biasing members 439 may be perpendicularly attached to the rims 435, 436. That is, even though the biasing members have a bend that protrudes inwardly to a center axis, the biasing members may still meet and be connected generally orthogonal at the rim. According to various embodiments, both the connector block and the spring are made from a same conductive material, which may assure conductivity and avoid ionic corrosion. For example, the connector block and the spring may be manufactured with a biocompatible material such as MP35N or Titanium (Grade 23).

The rims 435 and 436 may be have the shape of an arc. FIG. 4C generally illustrates an angle of rotation of the arc-shaped rim, and thus an angle of rotation of a gap 438 between the rim ends 437 of the arc-shaped rims 435, 436. The arc-shaped rim may have an angle of rotation greater than 240 degrees such that the gap has an angle of rotation less than 120 degrees. Each of the first and second rims may have an angle of rotation which may be greater than 270 degrees and the gap may have an angle of rotation less than 90 degrees. Each of the first and second rims may have an angle of rotation which may be greater than 315 degrees and the gap may have an angle of rotation less than 45 degrees. Each of the first and second rims may have an angle of rotation between 320 to 340 degrees and the gap may have an angle of rotation between 20 to 40 degrees.

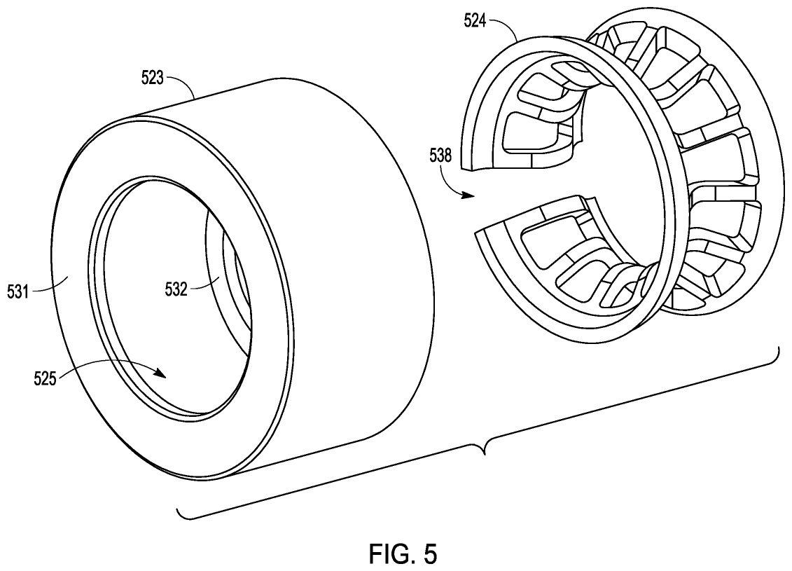

FIG. 5 illustrates, by way of example and not limitation, an assembly of a spring 524 into a connector block 523 to form a connector contact assembly. The spring 524 may be compressed, due to the shape memory of the material and the gap 538 which closes under compression, and then positioned with the cavity or open center portion 525 of the connector block 523. Assembling the connector contact assembly may include applying a compressive force to the spring 524 to reduce a spring size to a compressed size, inserting the spring 524 having the compressed size through an opening in one of the flanges 531, 532 so that the spring is with the interior of the connector block, and removing the compressive force, allowing the spring 524 to expand from the compressed shape to at least a partially expanded shape larger than the opening such that the spring 524 is retained within the interior of the connector block 523

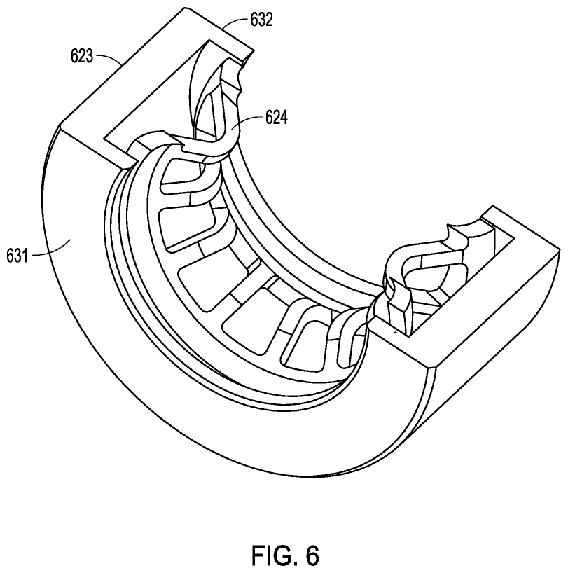

FIG. 6 illustrates, by way of example and not limitation, a cutaway view of the assembled contact connector. The expanded spring 624 is seated between the two flanges 631, 632. However, as the spring 624 is not welded or otherwise affixed within the connector block, the spring 624 is allowed some movement within the connector block 623 while still being retained within the connector block 623.

Notably, the spring and the connector block are separate parts of the assembly and have no permanent joint, allowing the spring to follow the lead profile, while the connector block retains the spring between the flanges of the connector block. This design allows the spring to position itself concentrically with the contour of the lead body. A benefit is that the contacting elements of the spring will not be stretched from misalignment during lead insertion. A same lead insertion force will be maintained after several lead insertions.

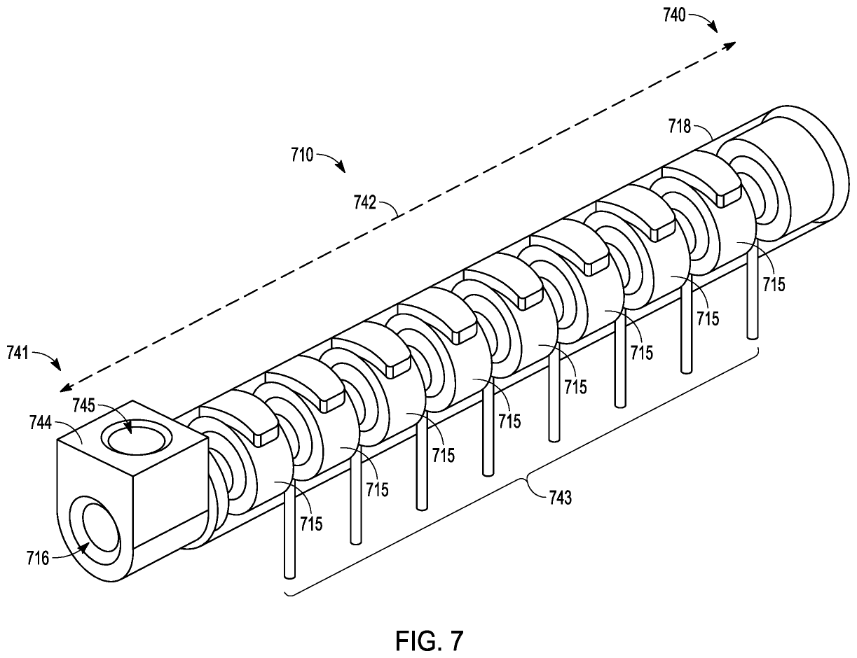

FIG. 7 illustrates, by way of example and not limitation, a connector 710 with eight connector contact assemblies 715. It is noted that contacts may be disposed in various types of connectors that, in turn, may disposed along various types of implantable medical devices including, for example, control modules, lead extensions, adaptors, splitters, or the like. The contacts are disposed in contact housings that are arranged along the connector and open to a connector lumen suitable for receiving an elongated member.

The connector 710 can be disposed, for example, on a control module, lead extension, adaptor, splitter, or the like. The connector 710 has a first end 740, an opposing second end 741, and a longitudinal length shown by a dashed line 742. The connector 710 may include an elongated connector housing 718 that defines a connector lumen 716 suitable for receiving a portion of an elongated member, such as a lead, lead extension, or the like. The connector lumen 716 is defined along the second end 741 of the connector 710 and extends along the longitudinal length 742 of the connector 710. The first end 740 of the connector 710 can be either open or closed.

Multiple connector contact assemblies 715 (illustrated as eight) are disposed in a spaced-apart relationship along the longitudinal length 742 of the connector housing 718 such that the connector contact assemblies 715 are exposed to the connector lumen 716 and also to an array of conductive members 743. When, for example, the connector 710 is disposed on a lead extension, the conductive members 743 may couple the connector contact assembly 715 to lead extension terminals. When, for example, the connector 710 is disposed on a control module, the conductive members 743 may couple the connector contact assembly 715 to the electronic subassembly. In at least some embodiments, the conductive members 743 couple the connector contact assembly 715 to the electronic subassembly via feedthrough pins extending through a sealed housing.

A retention block 744 may be disposed along the connector 710, which may be used to assist in the retention of an elongated member (e.g., lead or lead extension) when the elongated member is inserted into the connector lumen 716. The retention block 744 may define a fastening aperture 745 configured to receive a fastener (e.g., a set screw, pin, or the like). In at least some embodiments, the fastener, when received by the fastener aperture 745, is configured to tighten against a portion of the elongated member (e.g., a retention sleeve) when the elongated member is inserted into the connector lumen 716.

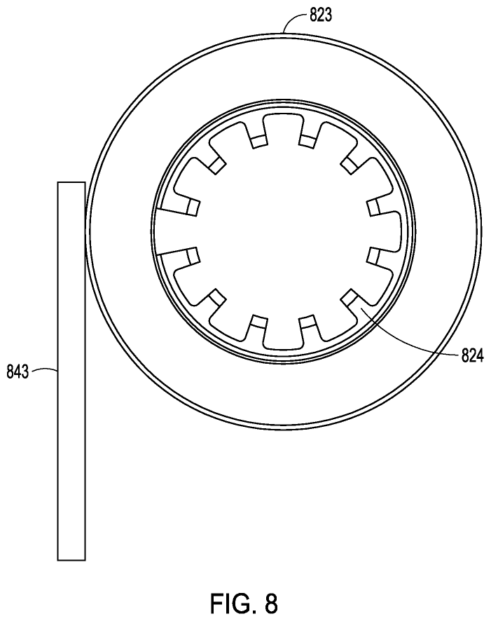

FIG. 8 illustrates, by way of example and not limitation, a conductive member 843 attached to the exterior of the conductor block 823 for use to provide an electrical connection to the spring 824. It is noted that the conductive member 843, the conductor block 823 and the spring 824 are all electrically conductive. Thus, the conductive member 843 may make an electrical connection with a lead terminal through the contact that the lead terminal makes with the spring 824, the contact that the spring 824 makes with the conductor block 823, and the contact between the conductor block and the conductive member 843.

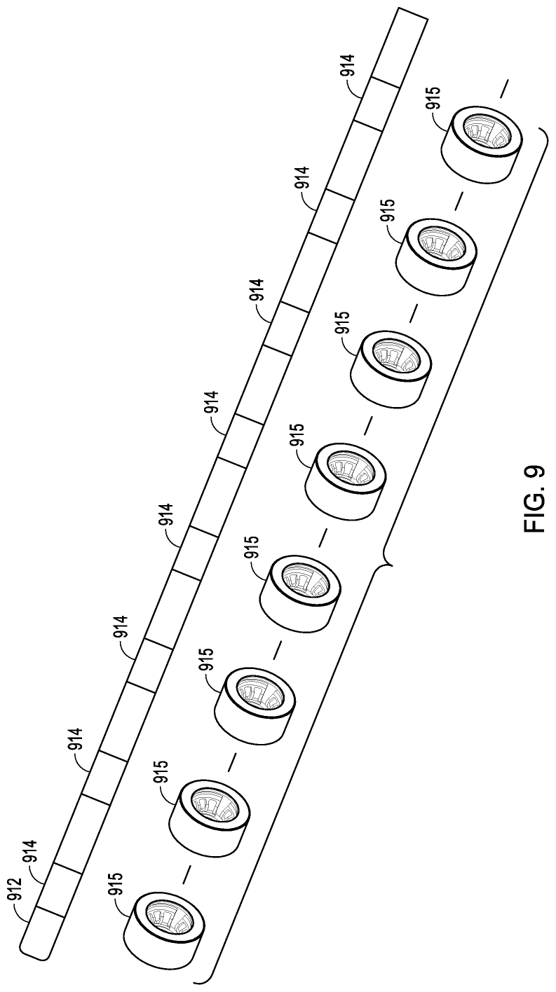

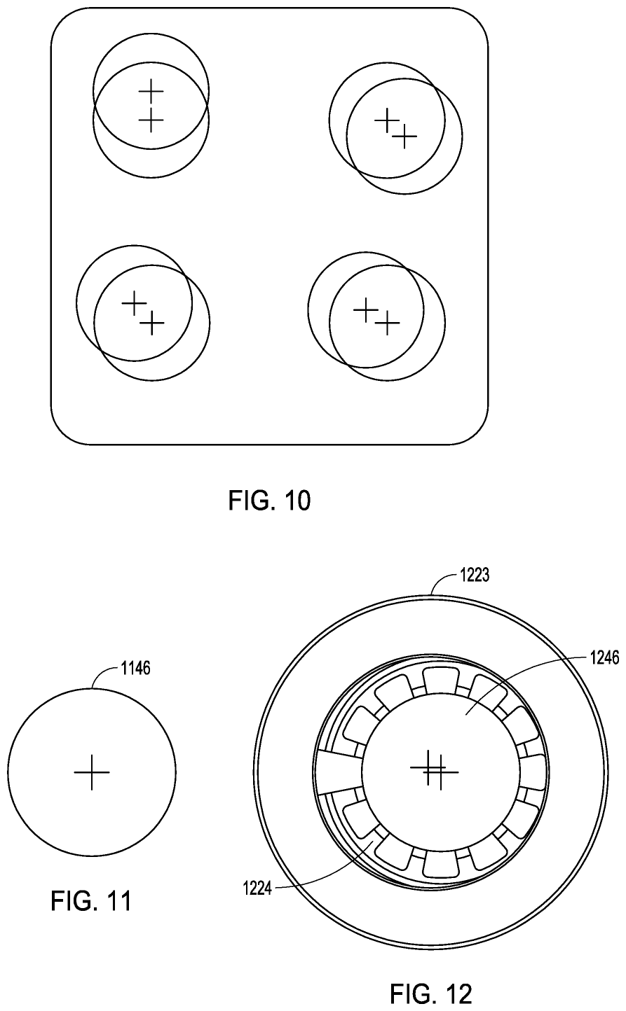

FIG. 9 illustrates a proximal end of a lead and a plurality of connector contact assemblies through which the lead is inserted. The proximal end of a lead body 912 may include a plurality of lead terminals 914. Also illustrated are similar connector contact assemblies 915 that correspond to the lead terminals 914. These connector contact assemblies 915 are positioned within connector housing (not shown in this figure). However, these similar connector contact assemblies may have some misalignment such that their individual center axis lines are not aligned the same line. The misalignment may be attributed to the tolerances for manufacturing and assembling components of the connector. This misalignment is illustrated in more detail below with respect to FIGS. 10-12.

FIG. 10 illustrates, by way of example and not limitation, a view from an end of the header having four lumens for receiving four leads into four connectors, and showing center offsets for the center axis (illustrated using “+”) of at least two of the connector blocks used in the connector contact assemblies for each connector. It can be seen that, when viewed in line, the connector blocks may not be aligned because of manufacturing tolerances. However, if such offsets were not compensated by the present subject matter, the insertion of the lead body into such offsets may induce undesired forces onto contacts within the connector. As identified earlier, the spring and the connector block are separate parts of the assembly and have no permanent joint, allowing the spring to follow the lead profile when the lead is inserted.

FIG. 11 illustrates, by way of example and not limitation, a lead with a center axis and a circular lead profile 1146. FIG. 12 illustrates, by way of example and not limitation, an offset between the center axes of a connector block 1223 and lead 1246 as illustrated by the two instances of “+”, and the accommodation of the offset by movement of the spring 1224 into alignment with the lead 1246. The spring is able to have some movement as it is retained between the flanges of the connector block. The connector block has a connector block center axis and the spring has a spring center axis generally parallel to the connector block when the spring is retained within the open center portion of the connector block. The movement concentrically positions the spring around the contour of the lead body, offsetting the spring center axis with respect to the connector block axis to accommodate the lead. Therefore, as illustrated in FIG. 12, the spring can follow the lead profile as the lead is inserted, such that the spring positions itself concentrically with the contour of the lead body. A benefit is that the contacting elements of the spring will not be stretched from misalignment during lead insertion, and a same lead insertion force will be maintained after several lead insertions. According to various embodiments, the spring may be positioned within approximately +/−0.005″ offset from center, such that the spring may accommodate a +/−0.005″ offset to the center line of the lead.

FIGS. 13A-13C illustrate, by way of example and not limitation, potential spring motion within the connector block to accommodate the offset. This arrangement permits the spring 1324 to have some movement within the connector block 1323 to accommodate the position and contour of lead body. This way the spring can position itself concentrically with the lead body contour and it will not accidentally ‘stretch’ the contacts within the connector. FIGS. 13A and 13B illustrate that there is some allowed up and down movement of the spring within the open center portion of the connector block, as well as some allowed back and forth movement in line with the lead body. FIG. 13C illustrates that the spring has some allowed movement within the open center portion of the connector block in all directions generally orthogonal to the center line axis of the connector block. The spring and connector blocks are designed to provide sufficient movement to accommodate slight differences in the alignment of connector blocks due to manufacturing tolerances.

Some benefits of the present subject matter include improved structural integrity as prevents or reduces stretch in contacts within the connectors, provides simpler construction, and reduces manufacturing costs by eliminating steps like connector spring welds. The connector of the present subject matter may be used for a variety of electrical connections, including but not limited to connecting leads to medical devices, such as neuromodulation leads. Examples of neuromodulation leads include, but are not limited to, leads used to deliver spinal cord stimulation (SCS), deep brain stimulation (DBS), peripheral nerve stimulation (PNS), functional electrical stimulation (FES), and transcutaneous electrical nerve stimulation (TENS).

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. Also contemplated are examples in which only those elements shown or described are provided, and examples using combinations or permutations of those elements shown or described.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A system for making an electrical connection to a lead contact of a medical device lead or lead extension, the system comprising a connector contact assembly including:
a connector block configured to receive and retain a conductive spring within an interior of the connector block to form the contact connector, wherein a combination of the connector block and the spring are configured to receive a proximal end of the medical lead or a proximal end of the lead extension when the proximal end is inserted through the combination;
the connector block having an open center portion, and further having a first end, an opposing second end, a longitudinal length extending between the first end and the second end, an outer surface, and an inner surface forming a perimeter of the open center portion of the connector block, the connector block further including a first flange at the first end and a second flange at the second end, wherein the first and second flanges extend inward from the inner surface of the connector block; and
the spring being formed from a material with shape-memory, the spring being configured to be compressed for insertion through one of the first flange or the second flange, and to at least partially expand into an expanded state such that the spring is retained between the first flange and the second flange within the open center portion of the connector block, the spring having a biasing structure configured to physically contact the lead contact when the proximal end is inserted into a final position within the connector contact assembly.

2. The system of claim 1, wherein both the connector block and the spring are conductive and in contact with each other, and a conductive member extends from an exterior of the connector block to provide an electrical connection to the spring retained within the open center portion of the connector block.

3. The system of claim 1, wherein the spring is not affixed to the connector block, and the combination of the connector block and the spring are configured to allow movement of the spring, when the spring is in the expanded state between the first flange and the second flange, within the open center portion of the connector block.

4. The system of claim 3, wherein, when the spring is retained within the open center portion of the connector block, the connector block has a connector block center axis and the spring has a spring center axis generally parallel to the connector block, and the movement moves an offset of the spring center axis with respect to the connector block axis to accommodate the lead.

5. The system of claim 4, wherein the spring includes
a first end and an opposing second end; and
a first rim at the first end and a second rim at the second end,
wherein each of the first and second rims have rim ends and a gap separating the rim ends,
wherein the biasing structure includes a plurality of biasing members configured to physically contact that lead contact, each of the plurality of biasing members extending between the first rim and the second rim and including a bend to provide a biasing force against the lead contact, and
wherein the biasing forces corresponding to the plurality of biasing members align the spring center axis with a center axis of the proximal portion of the lead or the lead extension.

6. The system of claim 5, wherein each of the plurality of biasing members are perpendicularly attached to the first rim and to the second rim.

7. The system of claim 5, wherein the first and second rims are arc-shaped.

8. The system of claim 5, wherein each of the first and second rims has an angle of rotation greater than 240 degrees and the gap has an angle of rotation less than 120 degrees.

9. The system of claim 5, wherein each of the first and second rims has an angle of rotation greater than 270 degrees and the gap has an angle of rotation less than 90 degrees.

10. The system of claim 5, wherein each of the first and second rims has an angle of rotation greater than 315 degrees and the gap has an angle of rotation less than 45 degrees.

11. The system of claim 5, wherein each of the first and second rims has an angle of rotation between 320 to 340 degrees and the gap has an angle of rotation between 20 to 40 degrees.

12. The system of claim 1, wherein both the connector block and the spring are made from a same conductive material.

13. The system of claim 1, wherein the biasing structure comprises at least one bend that extends into the open center portion of the contact housing and narrows an inner diameter of the open center portion.

14. The system of claim 1, wherein the connector block has a cylindrical shape, where both the outer surface and the inner surfaces are curved surfaces.

15. The system of claim 1, wherein the connector-contact assembly is one of a plurality of similar connector-contact assemblies, and the system further includes:
an elongated connector housing having a first end and an opposing second end;
a connector lumen defined in the connector housing, the connector lumen configured and arranged to receive the proximal end of the lead or the lead extension; and
the plurality of similar connector-contact assemblies.

16. The system of claim 15, wherein:
when the spring is retained within the interior of the connector block, the connector block has a connector block center axis and the spring has a spring center axis generally parallel to the connector block, and the movement moves an offset of the spring center axis with respect to the connector block axis to accommodate the lead;
at least two connector blocks within the plurality of connector-contact assemblies have connector block axes misaligned with respect to each other; and
the combination of the connector block and the spring for each of the plurality of plurality of connector-contact assemblies are configured to allow movement of the spring in the expanded state within the open center portion of the connector block when the spring is retained between the first flange and the second flange such that a spring axis align with a center axis of the proximal end of the lead or the lead extension.

17. A medical device, comprising:
a control module configured to be electrically connected to a lead,
the lead including:
a lead body with a proximal portion, a distal portion, and a longitudinal length,
a plurality of electrodes disposed along the distal portion of the lead body,
a plurality of terminals disposed along the proximal portion of the lead body, and
a plurality of lead conductors electrically coupling the plurality of electrodes to the plurality of terminals;
the control module including:
a housing;
an electronic subassembly disposed in the housing;
an elongated connector housing having a first end and an opposing second end;
a connector lumen defined in the connector housing, the connector lumen configured and arranged to receive the proximal end of the lead or the lead extension; and
a plurality of similar connector-contact assemblies, each of the plurality including:
a connector block configured to receive and retain a conductive spring within an interior of the connector block to form the contact connector, wherein a combination of the connector block and the spring are configured to receive a proximal end of the medical lead or a proximal end of the lead extension when the proximal end is inserted through the combination;
the connector block having an open center portion, and further having a first end, an opposing second end, a longitudinal length extending between the first end and the second end, an outer surface, and an inner surface forming a perimeter of the open center portion of the connector block, the connector block further including a first flange at the first end and a second flange at the second end, wherein the first and second flanges extend inward from the inner surface of the connector block; and
the spring being formed from a material with shape-memory, the spring being configured to be compressed for insertion through one of the first flange or the second flange, and to at least partially expand into an expanded state such that the spring is retained between the first flange and the second flange within the open center portion of the connector block, the spring having a biasing structure configured to physically contact the lead contact when the proximal end is inserted into a final position within the connector contact assembly.

18. A method, comprising:
assembling a connector contact assembly for use to make an electrical connection to a lead contact of a medical device lead or lead extension, wherein the connector contact assembly includes a connector block configured to receive and retain a conductive spring having shape memory within an interior of the connector block, and the connector block having a first open end, an opposing second open end, a longitudinal length extending between the first open end and the second open end, an inner surface, an outer surface, a first flange at the first open end and a second flange at the second open end where the first and second flanges extend past the inner surface toward the interior of the connector block, wherein the assembling the connector contact assembly includes:
applying a compressive force to the spring to reduce a spring size to a compressed size;
inserting the spring having the compressed size through an opening in a first flange or opening in a second flange so that the spring is with the interior of the connector block; and
removing the compressive force, allowing the spring to expand from the compressed shape to at least a partially expanded shape larger than the opening to the first flange or the second flange such that the spring is retained within the interior of the connector block.

19. The method of claim 18, wherein the spring is not affixed to the connector block when retained within the interior of the connector block.

20. The method of claim 18, wherein a combination of the connector block and the spring are configured to receive a proximal end of the medical lead or a proximal end of the lead extension when the proximal end is inserted through the combination.
